(19) United States
(12) Patent Application Publication
(10) Pub. No.: US 2006/0128961 A1
(43) Pub. Date: Jun. 15, 2006

(54) Process for the production of meso-substituted cyanine dyes

(75) Inventors: Hans-Joachim Timpe, Osterode/ Harz (DE); Eduard Kottmair, Ebenhausen (DE)

Correspondence Address: BETH READ;PATENT LEGAL STAFF, EASTMAN KODAK COMPANY, 343 STATE STREET, ROCHESTER, NY, 14650-2201, US

(73) Assignee: Kodak Polychrome Graphics LLC, Norwalk, CT (US)

(21) Appl. No.: 10/537,389

(22) PCT Filed: Dec. 4, 2003
(86) PCT No.: PCT/EP03/13727

§ 371 Date: Oct. 31, 2005

(30) Foreign Application Priority Data
Dec. 6, 2002 (DE) 102 57 188.0

(51) Int. Cl. C07D 417/02; C07D 413/02; C07D 403/02

(52) U.S. Cl. 546/153; 548/156; 548/219; 548/305.4

(57) ABSTRACT

A single-step process for the production of meso-substituted cyanine dyes is provided comprising the reaction of a dye (III), with (a) a methylene compound (IV) or (V) or a quaternary salt (VI) or (VII) and (b) a compound selected from functionalized (hetero)aromatic compounds Ar—B, saturated cyclic amines (IV) and heteroaromatic compounds (V).

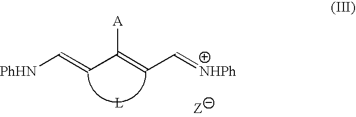

The present invention relates to a process for the production of meso-substituted cyanine dyes, in particular a single-step process that provides high yields of a meso-substituted cyanine dye of high purity.

Progress in the field of laser technology, in particular with respect to IR lasers, has led to an increased use of such lasers for a variety of applications. One area in which the use of lasers has strongly increased in the past few years are image-recording processes such as e.g. the production of printable images on printing plate precursors, in particular lithographic printing plate precursors, and the production of patterns on printed circuit boards.

The radiation-sensitive elements that are used in the production of printing plates, circuit boards and storage media such as e.g. DVDs and the like comprise a radiation-sensitive coating on a substrate. For the production of images/patterns or the storage of information, the coating is subjected to image-wise radiation that causes an image-wise modification of the coating; in a subsequent developing step, either the irradiated areas (in the case of positive working coatings) or the non-irradiated areas (in the case of negative working coatings) are removed from the printing plates, circuit boards and the like.

In radiation-sensitive elements that are imaged by means of an IR laser or on which information is stored, the coating comprises a substance that converts IR radiation to heat, which then causes a modification of the coating. This conversion substance strongly influences the radiation sensitivity of the coating so that in the last few years increased efforts have been undertaken to find conversion substances exhibiting as high a light absorption in the IR range as possible and a high degree of photothermal conversion efficiency, as well as good compatibility with the other components of the radiation-sensitive coating.

Polymethine dyes such as e.g. cyanine dyes and merocyanine dyes are a class of frequently used conversion substances.

EP-A-1 006 116 describes cyanine dyes with the following structure 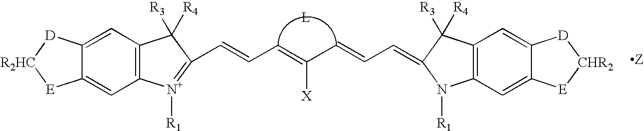
wherein X can be a hydrogen atom, a halogen atom or a substituted amino group.

These cyanine dyes absorb in the range of 750 to 900 nm and are therefore suitable for IR irradiation. They are produced by mneans of a condensation reaction of an indolenine compound and a diformyl compound or dianil compound in the presence of a fatty acid salt and an anhydride of an organic acid.

EP-A-1 188 797 discloses polymethine dyes with the following structure 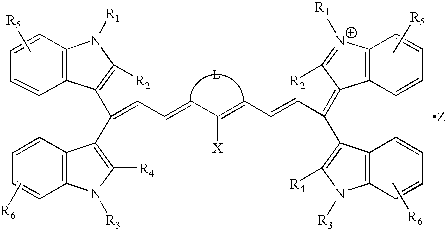
(wherein X is a hydrogen atom, a halogen atom or a substituted amino group), which absorb in the range of 900 to 1100.

They are prepared by means of a condensation reaction of a di-indolylethylene compound and a diformyl compound or a dianil compound in the presence of an alkali metal salt and an anhydride of an organic acid.

Additional cyanine dyes are described e.g. in EP-A-1 223 196. A group of dyes described in this document comprises a cyclopentenyl ring substituted with a diphenylamino group as meso-substituent. These cyanine dyes are synthesized by reacting the corresponding indolium iodide with an N-(2,5-bis((phenylamino)methylene)cyclopentylidene)-N-phenylbenzene-aminium salt in the presence of acetic acid anhydride and a trialkylamine.

However, the availability of diformyl compounds for the synthesis of cyanine dyes is limited. Such products are difficult to produce and decompose within a few hours under common storage conditions. For this reason they are not commercially available and have to be prepared immediately before any planned production of cyanine dyes.

A production of cyanine dyes that makes good economic sense therefore starts with blocked diformyl compounds which are available from the well-known Vilsmeier synthesis from ketones (in the presence of phosphoroxychloride or phosphoroxybromide, dimethyl-formamide and amines). Advantageously, aniline is used as amine. However, the use of such inexpensive raw materials leads to cyanine dyes comprising a chlorine or bromine substituent in the meso-position.

While cyanine dyes with a meso-Cl or meso-Br substituent exhibit good light-heat-conversion properties, they lead to unstable coatings in some coating compositions due to the reactive halogen atoms (which react for example with novolak). Other necessary properties required for different applications such as e.g. the location of the absorption maximum or thermal decomposability (which is important e.g. for their use in DVDs) can be modified by the substituents in the meso-position. Cyanine dyes with a meso-substituent selected from —S-aryl, —O-aryl, —Se-aryl, —SO₂-aryl, —NR—Ar, —(N-heterocycle), —S-heterocycle, —O-heterocycle or —NR-heterocycle are usually prepared by means of a two-step process; cf. Zollinger, “Color Chemistry”, 1991 (2^{nd} edition), p. 65-69. In a first step, a dye with a meso-chlorine substituent is prepared, which is then replaced in the second step with, e.g., —S-aryl. The advantages of this two-step synthesis are that the raw materials are easily accessible and inexpensive. In contrast, potential raw materials already containing the future meso-substituent can sometimes only be obtained by means of multi-step syntheses. Furthermore, by appropriately selecting the meso-substituent, the absorption properties of the obtained cyanine dye can be influenced as desired without the necessity of modifying the parent structure of the dye (variations over a range of about 80 nm are possible). Moreover, cyanine dyes with the mentioned meso-substituents are less reactive and thus lead to more stable coatings.

Reaction scheme (I) illustrates this two-step synthesis using a cyanine dye with a meso-phenylthio substituent (dye C) as an example.

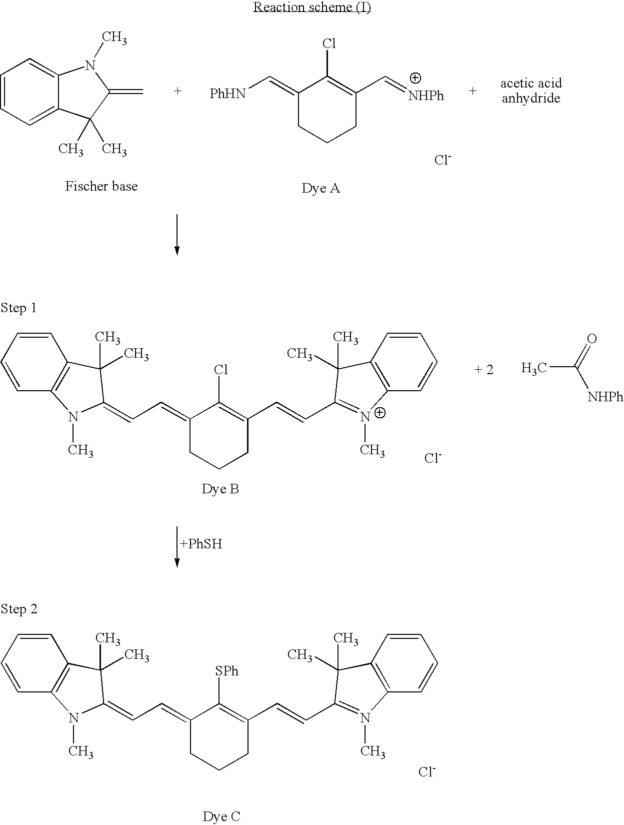

In the first step, aniline is formed as a side-product. This aniline has to be trapped with the help of acetic acid anhydride or other acid anhydrides since otherwise it would replace the meso-chlorinIe substituent in some cases and a meso-aniiine-substituted dye D would be formed as a side-product.

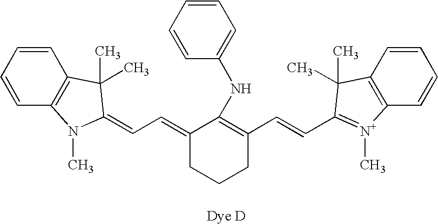

Thus, the dye B formed in the first step as a desired product is either contaminated with dye D (if no acid anhydride was added) or acetanilide has to be removed (if an acid anhydride was added). The formation of dye D in the first step leads to a marked decrease in the yield of desired dye B (and is furthermore difficult to remove due to its very similar structure) so that in practical applications an acid anhydride is usually added.

However, in order to obtain as pure a dye C as possible, the formed acetanilide has to be removed either after the first or the second step, which also significantly affects the yield of desired dye C.

Since due to their excellent properties as light-heat converters in radiation-sensitive coatings there is a wide-spread interest in cyanine dyes with meso-substituents different from Cl and Br, it is the object of the present invention to provide a process for the production of meso-substituted cyanine dyes (wherein the meso-substituent is different from Cl and Br), which starts with the inexpensive Vilsmeier adducts and provides, in an uncomplicated manner, high yields of high-purity meso-substituted cyanine dyes.

This object is achieved by a process for the production of cyanine dyes of the following formula (I) or (II) 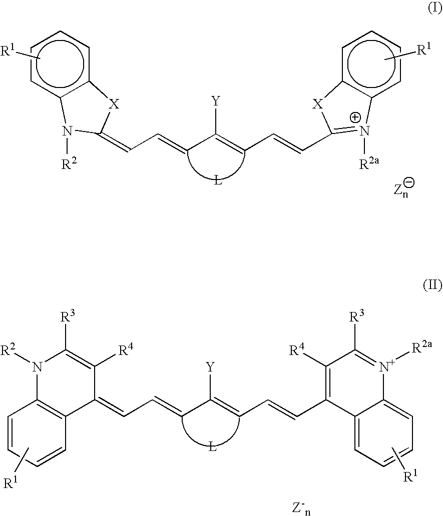
wherein
each R¹ is independently selected from —COOH, —SO₃H, a hydrogen atom, an optionally substituted C₁-C₁₂ alkyl, halogen, optionally substituted C₁-C₁₂ alkoxy, —NO₂, —CN and fused aromatic and heteroaromatic ring systems,
each X independently represents —CR³═CR⁴—, —O—, —S—; —NR⁶- or —CR⁵₂—,
R² is an optionally substituted C₁-C₁₂ alkyl, —(C₁-C₁₂ alkanediyl)-SO₃H, —(C₁-C₁₂ alkanediyl-)COOH or an optionally substituted aryl,
R^{2a} is an optionally substituted C₁-C₁₂ alkyl, —(C₁-C₁₂ alkanediyl)-SO₃⁻, —(C₁-C₁₂ alkanediyl)-COO⁻, —(C₁-C₁₂ alkanediyl)-NR⁶₃⁺ or an optionally substituted aryl,
R³and R⁴ are independently selected from —COOH, —SO₃H, —COOR⁵, —CN, —NO₂, —OH, —NR⁶₂, a hydrogen atom, an optionally substituted C₁-C₁₂ alkyl, an optionally substituted C₁-C₁₂ alkoxy, halogen and aryl,
jedes R⁵ is independently C₁-C₁₂ alkyl,
Z³¹ is selected from Cl⁻, Br⁻, I⁻, SCN⁻, PF₆⁻, SbF₆⁻, AsF₆⁻, aryl-SO₃⁻, alkyl-O—SO₃⁻, PO₄H₂⁻, CH₃SO₃⁻, CF₃SO₃⁻, (CF₃SO₂)₂N⁻, HSO₄⁻, BF₄⁻ and ClO₄⁻,
n is 0 if R^{2a} is —(C₁-C₁₂ alkanediyl)- SO₃⁻ or —(C₁-C₁ alkanediyl)-COO⁻,
n is 1 if R^{2a} is an optionally substituted C₁-C₁₂ alkyl or aryl,
n is 2 if R^{2a} is —(C₁-C₁₂ alkanediyl)-NR⁶⁺₃,
Y is selected from —S—Ar, —Se—Ar—, —O—Ar, —NR⁶—Ar, —SO₂—Ar and —(N-heterocycle).
R⁶ is a hydrogen atom or an optionally substituted C₁-C₁₂ alkyl,
Ar is an aromatic group wherein one or more ring carbon atoms are optionally replaced with heteroatoms selected from N, O and S, and 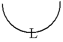
   represents C₂-C₃ alkanediyl, optionally comprising one or more substituents selected from C₁-C₁₀alkyl, aryl, halogen atoms and C₁-C₁₀alkoxy,
   said process comprising the single-step reaction of the dye of formula (III) 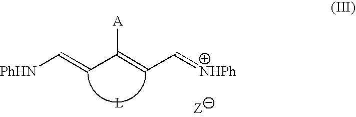
   wherein A is selected from Cl and Br and 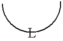
   is as defined above for formula (I), with
   (a) a compound selected from
   (i) a methylene derivative of formula (IV) or (V) 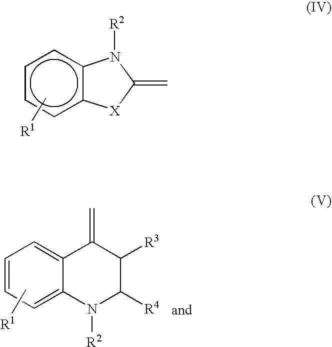
   (ii) a quaternary salt of formula (VI) or (VII), 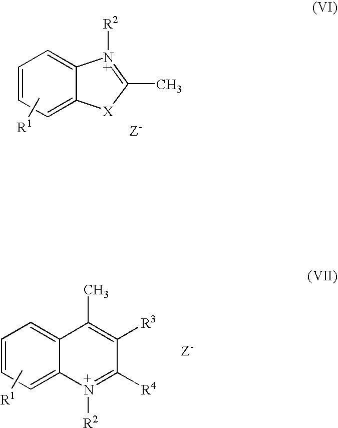
      wherein X, R¹, R², R³, R⁴ and Z are as defined in formulas (I) and (II), and
      (b) a compound selected from
   (i) aromatic and heteroaromatic functionalized compounds Ar—B,
   (ii) saturated 5- or 6-membered cyclic amines 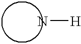
      and
   (iii) 5- or 6-membered heteroaromatic compounds 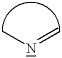
      comprising at least one nitrogen atom as heteroatom in the aromatic ring, which nitrogen atom is bonded to the two adjacent ring carbon atoms via a single and a double bond and furthermore comprises a free electron pair
      wherein
Ar represents a 5- or 6-membered aryl group, wherein one or more ring carbon atoms are optionally replaced with heteroatoms selected from N, O and S,
B is selected from —NHR⁶, —SH, —OH, —SeH and —SO₂H,
R⁶ is a hydrogen atom or an optionally substituted C₁-C₁₂ alkyl and the saturated cyclic amines optionally comprise an additional heteroatom selected from N, O and S in the ring,
in an inert organic solvent miscible with water.

Unless defined otherwise, the terms “alkyl” or “alkyl unit of an alkoxy group” as used in the present invention refers to a straight-chain, branched or cyclic saturated hydrocarbon group which optionally comprises one or more substituents selected from —OH, halogen and —CN. The same analogously applies to the term “alkanediyl”.

Unless defined otherwise, the term “aryl” as used in the present invention refers to a 5- or 6-membered aromatic carbocyclic group in which one or more ring carbon atoms can be replaced with heteroatoms selected from N, S and O (“heteroaromatic group”), and which can comprise one or more substituents bonded to ring carbon atoms and selected from —COOH, —SO₃H, —COOR⁵, —CN, —NO₂, —OH, —NR⁶₂, an optionally substituted C₁-C₁₂ alkyl, C₁-C₁₂ alkoxy and halogen; also, two or more aromatic 5-membered and/or 6-membered rings (carbocylic and/or heterocyclic) can be present in a fused ring system.

The term “—(N-heterocycle)” indicates that it is a heteroaromatic or saturated heterocyclic group comprising a nitrogen atom as heteroatom (and optionally additional heteroatoms selected from N, S and O), wherein this nitrogen atom is the binding site of the group. In other words, this group is derived from a saturated cyclic amine 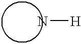
which optionally comprises additional heteroatoms in the ring, or from a 5- or 6-membered heteroaromatic compound 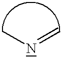
with a ring nitrogen atom bonded to the two adjacent ring carbon atoms via a single and a double bond and furthermore comprising a free electron pair.

Fused aromatic ring systems are e.g. fused benzene rings or fused naphthalene rings. These fused rings can optionally comprise one or more substituents as defined above for aryl groups. Examples of fused heteroaromatic ring systems include for example fused pyridine rings, pyrimidine rings, pyridazine rings and pyrazine rings.

In the process according to the present invention, a dye of formula (III) 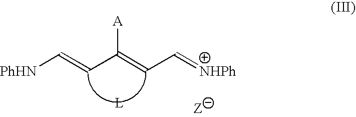
is used as one of the raw mnaterials.

The leaving group A is selected from Cl and Br; Cl is preferred since dyes of formula (III) wherein A=Cl can easily be produced and some dyes of formula (III) wherein A=Cl and Z⁻=Cl⁻ are commercially available.

In the formula, 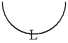
represents C₂-C₃ alkanediyl optionally comprising one or more substituents selected from C₁-C₁₀ alkyl (preferably methyl), C₁-C₁₀ alkoxy, aryl and halogen.

The counterion Z⁻ is selected from Cl⁻, Br⁻, I⁻, SCN⁻, PF₆⁻, SbF₆⁻, AsF₆⁻, aryl-SO₃⁻, alkyl-O—SO₃⁻, PO₄H₂⁻, CH₃SO₃⁻, CF₃SO₃⁻, (CF₃SO₂)₂N⁻, HSO₄⁻, BF₄⁻, ClO₄⁻; Z⁻ is preferably Cl³¹ (however, it is not required that A and Z⁻ be derived from the same element).

It is possible to already select the anion Z desired for the end product (I) or (II) as Z⁻ in the compound of formula (III). Alternatively, another anion Z⁻ can be used and an anion exchange can be carried out in the obtained product.

Preferably, compound (III) is used as chloride salt since these salts can be obtained in high yields by means of a Vilsmeier synthesis using POCl₃.

In one embodiment, the second raw material is a methylene derivative of formula (IV) (if a cyanine dye of formula (I) is to be synthesized) or a methylene derivative of formula (V) (if a cyanine dye of formula (II) is to be synthesized).

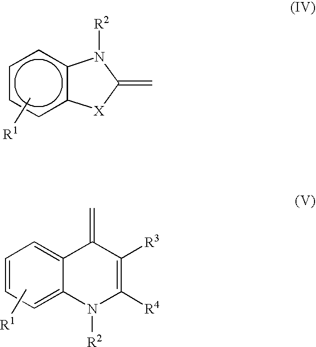

In another embodiment, a quaternary heterocyclic compound (VI) (for the synthesis of (I)) or compound (VII) (for the synthesis of (II)) is used instead of a methylene derivative of formula (IV) or (V), 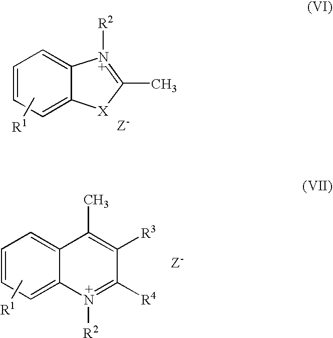
wherein X, R¹, R², R³, R⁴ and Z⁻ are as defined in formulas (I) and (II).

Preferably, R¹ is selected from a hydrogen atom, a fused benzene ring and a group —SO₃H.

R²/R^{2a} represents an optionally substituted C₁-C₁₂ ally, optionally substituted aryl, —(C₁-C₁₂ alkanediyl)-SO₃⁻/SO₃H, —(C₁-C₁₂ alkanediyl)-COO⁻/COOH or —(C₁-C₁₂ alanediyl)-NR⁵₃⁺, preferably —CH₃ or —(CH₂)₃SO₃H. The expression —SO₃⁻/SO₃H and —COO⁻/COOH indicates that both the acid and the acid anion are meant, If R^{2a} carries a negative charge, it compensates for the positive charge of the nitrogen atom so that no counterion Z⁻ is necessary (i.e. n=0). Accordingly, if R^{2a} carries a positive charge, two counterions Z³¹ are necessary (i.e. n=2).

X is selected from —CR³═CR⁴—, —O—, —S—, —NR⁵- and —CR₂⁵— and is preferably —C(CH₃)₂—, —S— or —N(CH₃)—.

R⁵ is C₁-C₁₂ alkyl, preferably —CH₃ or —CH₂CH₃.

R⁶ is a hydrogen atom or an optionally substituted C₁-C₁₂ alklyl, preferably H, —CH₃ or —CH₂CH₃.

If the cyanine dyes (I) or (II) to be produced exhibit a symmetrical structure, 2 moles of the methylene compound (IV) or (V) or of the quaternary salt (VI) or (VII) are reacted with 1 mole of compound (III). If the cyanine dyes (I) or (II) to be produced exhibit an unsymmetrical structure, 1 mole of a first methylene compound or of a first quaternary salt and 1 mole of a different second methylene compound or of a second quaternary salt is used together with 1 mole of compound (III).

In an especially preferred embodiment the cyanine dyes (I) or (II) exhitit a symmetrical structure, i.e. only one type of compounds (IV), (V), (VI) or (VII) is used.

The function of compound (b) is to replace the meso-substituent A with the desired substituent Y.

In the functionalized aromatic or heteroaromatic compound Ar—B, the functional group B can be bonded to any carbon atom of the aromatic or heteroaromatic. The compound Ar—B can comprise one or more functional groups B; in addition to substituent B, the group Ar can also comprise one or more other substituents (see the definition of aryl groups given above).

The aryl unit Ar can be a carbocyclic aromatic unit with 6-20 carbon atoms such as e.g. phenyl or naphthyl, or a heterocyclic aromatic unit with 5-20 ring atoms wherein at least one is different from carbon and selected from O, S and N, such as e.g. pyridine, thiophene, furan, pyrimidine, quinoline, imidazole, oxazole, thiazole, 1,2,4triazole, tetrazole, benzoxazole, naphtho[2,3-d]thiazole, benzimidazole or selenazole. If the functional group B is selected from —SH, —SeH and —SO₂H it is bonded to a ring carbon atom. If the functional group B is —NHR⁶ or —OH it is bonded to a ring carbon atom or, if it is an aromatic N-heterocycle, it is bonded to a ring carbon atom or the ring nitrogen atom.

If the compound Ar—B is aniline (i.e. Ar=phenyl; B=NH₂), further substituents can optionally be present at the phenyl ring as long as the substituted aniline is more nucleophilic than aniline itself. Optional substituents at the aniline include e.g. —N(Alkyl)₂, —O—aLkyl und —S-alkyl; 4-dimethylaminoaniline, 4-methoxyaniline and 4-phenylaminoaniline are preferred.

If the functional group Ar—B is not —NH₂, the aryl unit Ar can optionally comprise one or more additional substituents selected from halogen atoms, —NO₂, C₁-C₈ alkoxy, C₁-C₈ alkyl, —COOH, —SO₃H, —SCN, —COOR, —CN, —OH, —S—(C₁-C₈ alkyl) or —NR⁵₂. The positions of the substituents in relation to each other and in relation to the functional group B are only significant in that they do not bear too strong an influence on the reactivity of group B in particular due to steric effects. If the aryl unit comprises two chemically different groups B, it is the more nucleophilic group that preferably reacts. If the difference in nucleophilicity is large enough (e.g. B=—NH₂ and B=—SH or B=—NHR⁶ and B=—OH), only one cyanine dye of high purity is formed (in the examples mentioned above, —SH and —OH react, respectively).

If the aryl unit Ar— is an aromatic group with fused carbocyclic and heterocyclic portions, the functional group —B can be bonded either to the carbocyclic part or the heterocyclic part.

Instead of a functionalized (hetero)aromatic compound Ar—B, a saturated 5- or 6-membered cyclic amine 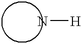
can be used as well, which can optionally comprise one or more further heteroatoms in the ring in addition to the amino nitrogen. The ring can optionally comprise one or more substituents selected from C₁-C₁₂ alkyl and C₁-C₁₂ alkoxy. Preferred cyclic amines are piperdine, piperazine, pyrrolidine and morpholine.

According to another embodiment, a 5- or 6-membered heteroaromatic compound 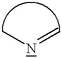
is used as compound (b), which comprises at least one nitrogen atom as heteroatom in the aromatic ring which is bonded to the two adjacent ring carbon atoms via a single and a double bond and furthermore comprises a free electron pair. The ring carbon atoms can optionally carry substituents selected from the substituents mentioned above for an aryl group; aromatic 5- or 6-membered rings (carbocyclic and/or heterocyclic), such as e.g. benzene rings, can also be fused to the aromatic N-heterocycle. If in addition to the nitrogen atom bonded to the two adjacent ring carbon atoms via a single and a double bond and comprising a free electron pair other heteroatoms are present in the ring, no hydrogen atom should be bonded to them; furthermore, no substituent as defined above for B should be present in the heteroaromatic compound 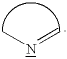
Examples of such a heteroaromatic compound 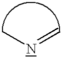
include pyridine, pyrimidine, pyrazine, 1,3,5- or 1,2,4-triazine, quinoline, N-substituted imidazole (e.g. N-methylimidazole) and N-substituted 1,2,4-triazole.

According to a preferred embodiment, (hetero)aromatic compounds Ar—B are used.

The reaction is carried out in an inert organic solvent, i.e. a solvent that does not react with the raw materials and the product. Furthermore, the solvent should be miscible with water in all proportions. Examples of suitable solvents include low alcohols (e.g. methanol, ethanol, n-propanol and iso-propanol), glycol ethers (e.g. ethylene glycol ether and propylene glycol ether), tetrahydrofuran, dioxane, acetone, dimethylformamide, dimethylacetamide, N-methylpyrrolidone, hexamethylphosphoric acid amide, acetonitrile and mixtures thereof; glycol ethers and low alcohols are especially preferred.

The reaction temperature is not particularly restricted and is preferably in the range of 15° C. to the boiling point of the solvent used. It is especially preferred to mix the reactants at room temperature and to not take measures such as active cooling to counter the heat generated by the exothermic reaction, but to let the reaction mixture cool by itself; carrying out the reaction in this manner is especially advantageous for economic reasons since no energy is required either for heating or for active cooling.

The reaction time is not particularly restricted; it obviously depends on the reactants and the reaction temperature. It is usually in the range of 10 minutes to 5 hours, preferably 0.5 to 2 hours.

The reaction vessel does not have to fulfill any specific requirements, either. Usually, the reaction mixture is stirred during the reaction, for which purpose conventional stirrers can be used.

The order in which the reactants are added is not particularly restricted. According to a preferred embodiment, the aromatic compound of formula (VIII) and the dye of formula (III) are provided in the reaction vessel and the methylene compounds or quaternary compounds of formulas (IV), (V), (VI) or (VII) are added in dissolved form. This way, especially pure cyanine dyes are obtained. If an asymmetric cyanine dye is to be produced, the following has to be kept in mind: The aromatic compound of formula (VIII) and the dye of formula (III) are provided in the reaction vessel and then the molar amount of the first methylene base or quaternary compound and base is added. After about 1 to 2 hours of reaction time, the molar amount of the second methylene base or the second quaternary compound is added.

The addition of alkali hydroxide (especially preferred NaOH or KOH) is recommended for Ar—B compounds wherein B equals —SH, —OH, —SeOH or —SO₂H in order to convert them to their more nucleophilic anion. This shortens the reaction time and increases the yield of the desired meso-substituted cyanine dye. If quaternary salts (VI) or (VII) are used as starting materials, it is advisable to add corresponding molar amounts of base to form the methylene bases from the salts.

The synthesized cyanine dye can be precipitated e.g. by the addition of an aqueous hydrochloric acid solution or sulfuric acid. Upon adding the corresponding molar amount of acid, the released aniline is converted to a water-soluble derivative; otherwise, slimy products are obtained that cannot easily be separated from the aqueous solution.

After isolation of the solid reaction product, for which all common isolation processes for solid substances from liquids (e.g. filtration, centrifugation) can be used, the moist product is dried. This drying step does not require any specific conditions, either.

In order to remove small amounts of any possibly formed side-products without yield loss, they are extracted from the dried reaction product with extracting agents. The decisive factor for the selection of the extracting agent is the solubility (or rather insolubility) of the cyanine dye in this agent in order to avoid yield loss. Especially suitable are solvents such as methyl ethyl ketone, methyl isopropyl ketone, acetic acid methyl ester, acetic acid ethyl ester, dioxane and tetrahydrofuran, in which cyanine dyes practically do not dissolve at all.

After extraction of the side-products the cyanine dye is again dried by means of common drying methods.

The cyanine dyes of formula (I) and (II) produced according to the process of the present invention preferably exhibit a purity of at least 98% after extraction. By means of an additional purification (e.g. recrystallization), purity is preferably increased to at least 99%, however, the yield decreases. The degree of purity obtained after extraction is completely sufficient for common applications of the dyes in photosensitive mixtures. The degree of purity given herein not only includes solid substances but also possible residual moisture and/or solvents; thus, the purity values with respect to solid substances alone would even be higher.

The anion Z⁻ of the thus prepared cyanine dye (I) or (II) can be replaced using known methods of anion exchange. For this purpose, it is advantageous to exploit the different solubility of cyanine dyes comprising the same organic group but different anions Z⁻. If a dye that due to its anion easily dissolves in a given solvent is to be converted to a less soluble dye, a solution of an ammonium or alkali salt comprising the desired anion Z⁻ is added to the solution of this dye.

### EXAMPLE 1

### Preparation of 2-[2-[2-thiophenyl-3-[2-(1,3-dihydro-1,3,3-trimethyl-2H-indole-2-ylidene)-ethyldene]-1-cyclohexene-1-yl]-ethenyl]-1,3,3-trimethyl-3H-indolinium chloride

110 g thiophenol and 360 g dye A (both available from Aldrich) were added under stirring to 2,000 ml 1-methoxy-2-propanol (Downanol PM) in a 10 l three-necked flask equipped with a stirrer and a reflux condenser. Then a solution of 40 g sodium hydroxide and 365 g 2-methylene-1,3,3-trmethylindoline (Fischer base, available from Aldrich) in 350 ml ethanol was added under stirring for one minute to this suspension. The reaction mixture warmed up to about 40° C. without the addition of external heat. Then the reaction mixture was left to cool to room temperature and 6 l of a 2 wt.-% hydrochloric acid were added. After the reaction mixture had cooled to room temperature, the insoluble portion was separated by filtration and washed with 2 l of water. Then the product was dried for one day at 50° C. in a circulating air oven. Yield: 556 g (94% based on dye A), IR dye content: 90 wt.-% (determined by measuring the optical density at 788 nm in methanol using an extinction coefficient ε of 435 l/g×cm for the pure IR dye), moisture content: 3.2 wt.-%.

The dried product was suspended in 5 l methyl ethyl ketone and heated to 80° C. for one hour. Subsequently, the solution with a temperature of about 40° C. was filtered and the solid portion was washed with ethyl acetate. The product was air-dried. Yield: 462 g (78 wt.-% based on dye A), IR dye content: 97.8 wt.-% (determined by measuring the optical density at 788 nm in methanol using an extinction coefficient ε of 435 l/g×cm for the pure IR dye), moisture content: 1.1 wt.-%, melting point: 222-223° C. (decomposition).

For determining whether an aniline-substituted dye D had been formed, a thin-layer chromatography was carried out with the following parameters:
TLC plates: Silica gel 60 F₂₅₄ (available from Merck)
Eluent: Solvent mixture of 60 vol.-% n-butanol, 20 vol.-% water, 10 vol.-% ethanol and 0.5 vol.-% acetic acid
3 samples (non-purified dye, purified dye and dye D (available from Hayashibara, Japan)) were dissolved in methanol and applied to the plate. After elution with the solvent mixture, the spot corresponding to dye D was not observed in the non-purified or in the purified dye samples.

### EXAMPLE 2

### Preparation of 2-[²-[²-(thio-5-thiomethyl-1,3,4-thiadiazole)-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride

16.4 g ²-mercapto-5-thiomethyl-1,3,4-thiadiazole (available from FEW, Wolfen/Germany) and 36.0 g dye A (available from Aldrich) were added under stiring to 200 ml Downanol PM in a 1 l three-necked flask equipped with a stirrer and a reflux condenser. Then a solution of 4.0 g sodium hydroxide and 36.5 g 2-methylene-1,3,3-trimethylindoline (Fischer base, available from Aldrich) in 35 ml ethanol was added under stirring for one minute to this suspension. The reaction mixture warmed up to about 40° C. without the addition of external heat. Then the reaction mixture was left to cool to room temperature and 600 ml of a 2 wt.-% hydrochloric acid were added. After the reaction mixture had cooled to room temperature, the insoluble portion was separated by filtration and washed with 2 l of water. Then the product was dried for one day at 50° C. in a circulating air oven. Yield: 56.2 g (92.9% based on dye A).

The dried product was suspended in 500 ml methyl ethyl ketone and heated to 80° C. for one hour. Subsequently, the solution with a temperature of about 40° C. was filtered and the solid portion was washed with ethyl acetate. The product was air-dried. Yield: 48.6 g (80.3 wt.-% based on dye A), UV/Vis spectrum in methanol: λₘₐₓ=796 nm, extinction coefficient ε=332 l/g×cm.

### EXAMPLES 3-9

### Preparation of 2-[2-[2-thio-substituted-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride

The syntheses were carried out as described in Example 2, with the exception that instead of 2-mercapto-5-thiomethyl-1,3,4-thiadiazole, 0.1 moles of the mercapto compounds listed in Table 1 were used. The yields and the UV/Vis spectroscopic data of the isolated cyanine dyes are listed in Table 1.

TABLE 1

| | | | UV/Vis data** | |
|---|---|---|---|---|
| | | Yield* | λₘₐₓ | Ext. coeff. |
| Example | Mercapto compound used | (wt. -%) | (nm) | (L/g × cm) |
| 3 | 4-Br—C₆H₄SH | 81.0 | 789 | 360 |
| 4 | 4-Cl—C₆H₄SH | 80.5 | 789 | 380 |
| 5 | 4-NH₂—C₆H₄SH | 77.5 | 786 | 387 |
| 6 | 4-NO₂—C₆H₄SH | 82.0 | 793 | 375 |
| 7 | 4-CH₃-5-mercapto- | 79.0 | 795 | 390 |
| | 1,2,4-triazole | | | |
| 8 | 1-C₆H₅-5-mercaptotetrazole | 80.5 | 800 | 336 |
| 9 | 1-CH₃-5-mercaptotetrazole | 80.0 | 798 | 377 |

*based on dye A
**measured in methanol

### EXAMPLE 10

### Preparation of 2-[2-[2-thiophenyl-3-[2-(1,3,3-dihydro-1,3,3-trimethyl-2H-benzo[e]indole-2-ylidene)-ethylidene]-1-cyclohexene-1-yl]-ethenyl]-1,3,3-trimethyl-1H-benzo[e]indolinium tosylate

The synthesis was carried out as described in Example 1, with the exception that 790 g 1,2,3,3-tetramethyl-benzo[e]indolinium tosylate (available from Eastman Kodak) were used instead of 2-methylene-1,3,3-trimethylindoline. The yield after clean-up in methyl ethyl ketone as 81 wt.-%; UV/Vis spectrum in methanol: λₘₐₓ=826 nm, extinction coefficient ε=282 l/g×cm.

### EXAMPLES 11-14

### Preparation of 2-[2-[2-aryloxy-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride

The synthesis was carried out as described in Example 1, with the exception that 0.1 moles of the phenols listed in Table 2 were used instead of the thiophenols. The yields after clean-up in methyl ethyl ketone and the UV/Vis spectroscopic data of the isolated cyanine dyes are listed in Table 2.

TABLE 2

| | | | UV/Vis data** | |
|---|---|---|---|---|
| | | Yield* | λₘₐₓ | Ext. coeff. |
| Example | Phenol used | (wt. -%) | (nm) | (L/g × cm) |
| 11 | phenol | 79.2 | 760 | 411 |
| 12 | 4-aminophenol | 65.5 | 762 | 403 |
| 13 | 4-methoxyphenol | 78.7 | 763 | 397 |
| 14 | 4-nitrophenol | 87.1 | 768 | 390 |

*based on dye A
**measured in methanol

### EXAMPLE 15

### 2-[2-[2-Phenylsulfonyl-3-[2-(1,3-dihydro-1,3,3-trimethyl-2H-indole-2-ylidene)-ethylidene]-1-cyclohexene-1-yl]-ethenyl]-1,3,3-trimethyl-3H-indolium chloride

16.4 g sodium salt of benzenesulfinic acid and 36.0 g dye A (both available from Aldrich) were added under stirring to 200 ml Downanol PM in a 1 l three-necked flask equipped with a stirrer and a reflux condenser. Then a solution of 4.0 g sodium hydroxide and 36.5 g 2-methylene-1,3,3-trimethylindoline (Fischer base, available from Aldrich) in 35 ml ethanol was added under stiing for one minute to this suspension. The reaction mixture was heated to 50° C. for one hour. Then the reaction mixture was left to cool to room temperature and 600 ml of a 2 wt.-% hydrochloric acid were added. After the reaction mixture had cooled to room temperature, the insoluble portion was separated by filtration and washed with 2 l of water. Then the product was dried for one day at 50° C. in a circulating air oven. Yield: 56.2 g (92.9% based on dye A).

The dried product was suspended in 450 ml methyl ethyl ketone and heated to 80° C. for one hour. Subsequently, the solution with a temperature of 40° C. was filtered and the solid portion was washed with ethyl acetate. The product was air-dried. The yield after clean-up was 82.5 wt.-%; UV/Vis spectrum in methanol: λₘₐₓ=828 nm, extinction coefficient ε=251 l/g×cm.

### EXAMPLES 16-18

### Ion exchange in 2-[2-[2-thiophenyl-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride

### Exanples 16 and 17

To a solution of 5.9 g 2-[2-[2-thiophenyl-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride in 60 g ethanol or Dowanol PM was added an equivalent amount of sodium iodide or sodium rhodanide in ethanol or Dowanol PM. The mixtures were added to 300 ml water, the precipitates were isolated by filtration, dried and recrystallized from ethanol or Dowanol PM. The yield after clean-up and the UV/Vis spectroscopic data of the isolated cyanine dyes are summarized in Table 3.

### Example 18

To a solution of 5.9 g 2-[2-[2-thiophenyl-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride in 60 g ethanol or Dowanol PM was added a solution of 1.84 g sodium hexafluorophosphate in 30 ml water. The formed precipitate was isolated by filtration, dried and recrystallized from ethanol. The yield after clean-up and the UV/Vis spectroscopic data of the isolated cyanine dye are given in Table 3.

TABLE 3

| | | | UV/Vis data** | |
|---|---|---|---|---|
| | | Yield* | λₘₐₓ | Ext. coeff. |
| Example | Salt used | (wt. -%) | (nm) | (L/g × cm) |

| | | | | |
|---|---|---|---|---|
| 16 | NAI | 85.3 | 788 | 370 |
| 17 | NaSCN | 77.1 | 788 | 405 |
| 18 | NaPF₆ | 93.2 | 788 | 355 |

*based on 2-[2-[2-thiophenyl-3-[2-(1,3-dihydro-1,3,3,-trimethyl-2H-indole-2-ylidene)-ethylidene]-1-cyclohexene-1-yl]-ethenyl]-1,3,3-trimethyl-3H-indolinium chloride
**measured in methanol

### EXAMPLE 19

### Ion exchange in 2-[2-[2-phenylsulfonyl-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride

A solution of 6.3 g 2-[2-[2-phenylsulfonyl-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride in 50 g ethanol was heated to 60° C. and a solution of 2.7 g sodium hexafluoroantimonate in 20 g ethanol was added. Upon cooling, a precipitate formed which was isolated by filtration, washed first with water and then with some cold ethanol and then dried. The yield was 95%.

The substitution with the sulfur atom of the sulfinic acid group was confirmed by X-ray examination (ENRAF-NONIUS CAD 4, single-crystal diffractometer). Needles that were needed for these examinations were prepared by dissolving the dye in dichloromethane and subsequent precipitation with diethylether.

### EXAMPLE 20

### 2-[2-[2-(4-Dimethylaminopyridinium)-3-[2-(1,3-dihydro-1,3,3-trimethyl-2H-indole-2-ylidene)-ethylidene]-1-cyclohexene-1-yl]ethenyl]-1,3,3-trimethyl-3H-indolium dichloride

3.6 g 4-dimethylaminopyridine and 11.8 g dye A (both available from Aldrich) were added under stirring to 60 ml Downanol PM in a 0.5 l three-necked flask equipped with a stirrer and a reflux condenser. Then 11.8 g 2-methylene-1,3,3-trimethylindoline (Fischer base, available from Aldrich) was added under stirring for one minute to this suspension. The reaction mixture was heated to 80° C. for 2 hours. Then the reaction mixture was left to cool to room temperature and 200 ml of a 1 wt.-% hydrochloric acid were added. After the reaction mixture had cooled to room temperature, the insoluble portion was separated by filtration and washed with 0.5 l of water. Then the product was dried for one day at 50° C. in a circulating air oven. Yield: 15.6 g (81.0 % based on dye A).

The dried product was suspended in 150 ml methyl ethyl ketone and heated to 80° C. for one hour. Subsequently, the solution with a temperature of about 40° C. was filtered and the solid portion was washed with ethyl acetate. The product was air-dried. The yield after clean-up was 78 wt.-%; UV/Vis spectrum in methanol: λₘₐₓ=786 nm, extinction coefficient ε=381 l/g×cm.

### COMPARATIVE EXAMPLE 1

### Two-step synthesis of 2-[2-[2-thiophenyl-3-[2-(1,3-dihydro-1,3,3-trimethyl-2H-indole-2-ylidene) -ethylidene]-1-cyclohexene-1-yl]-ethenyl]-1,3,3-trimethyl-3H-indolinium chloride

First step: Synthesis of 2-[2-[2-chloro-3-[2-(1,3-dihydro-1,3,3-trimethyl-2H-indole-2-ylidene)-ethylidene]-1-cyclohexene-1-yl]-ethenyl]-1,3,3-trimethyl-3H-indolinium chloride (dye B)

792 g dye A were added under stirng to 2,650 g acetic acid anhydride (both available from Aldrich) in a 30 l three-necked flask equipped with a stirrer and a reflux condenser. Then 770 g 2-methylene-1,3,3-trimethylindoline fischer base, available from Aldrich) was added under stirring for one minute to this suspension. Within one hour, the reaction mixture warmed up to about 50° C. After two more hours of stirng, 18 l water were added to the reaction mixture. Then the reaction mixture was left to cool to room temperature and 20 g sodium chloride were added. Subsequently, the insoluble portion was separated by filtration and washed with 2 l water. Then the product was dried for one day at 50° C. in a circulating air vacuum oven. Yield: 1,063 g (95% based on dye A), IR dye content: 72.0 wt-% (determined by measuring the optical density at 775 nm in methanol using an extinction coefficient for the pure IR dye of 500 l/g×cm), moisture content: 3 wt.-%.

The dried product was suspended in 2 l ethyl acetate and heated to 76° C. for one hour. Subsequently, the solution with a temperature of about 50° C. was filtered and the isolated product was air-dried. The remaining solution was strongly colored. The yield of solid product after the first purification step was 925 g (81 wt.-%) with a content of dye B of 80%.

The dried product was suspended in 1 l methyl ethyl ketone and heated to 80° C. for one hour. Subsequently, the solution with a temperature of about 40° C. was filtered and the isolated solid substance was air-dried. The yield after this second purification step was 705 g (62 wt.-%) with a purity of dye B of 89.2%. For further clean-up, the second purification step was repeated and 552 g dye B (48%) with a purity of 96.3% dye B were obtained (moisture content: 0.7 wt.-%).

Second step: Substitution of the meso-chlorine substituent by thiophenol

520 g dye B were added under stirring to 1 l ethanol in a 10 l three-necked flask equipped with a stirrer and reflux condenser. Subsequently, 110 g thiophenol (available from Aldrich) and then a solution of 40 g sodium hydroxide in 100 ml water were added within 5 minutes under stirring. Within one hour the reaction mixture warmed up to about 50° C. After another hour of stirring, 4.5 l water were added to the reaction mixture. Then the reaction mixture was left to cool to room temperature and the precipitated IR dye was separated by filtration and washed with 10 l water. Then the product was dried for one day at 60° C. in a circulating air oven. Yield: 595 g (95% based on dye B), IR dye content: 93.5 wt.-%, moisture content: 3.5 wt.-%.

The dried product was suspended in 3 l methyl ethyl ketone and heated to 80° C. for one hour. Subsequently, the solution with a temperature of about 40° C. was filtered and the isolated solid substance was air-dried. The yield after this purification step was 551 g (93 wt.-% based on dye B) and exhibited an IR dye purity of 95.1 % (moisture content: 2.1 wt.-%).

The total yield of IR dye based on the originally used dye A or Fischer base in this two-step synthesis was only 45 wt.-%. This value is much lower than the 78 wt.-% obtained in the single-step synthesis of Example 1 according to the present invention.

1-14. (canceled)

15. A process for the production of a meso-substituted cyanine dye represented by formulas (I) or (II) 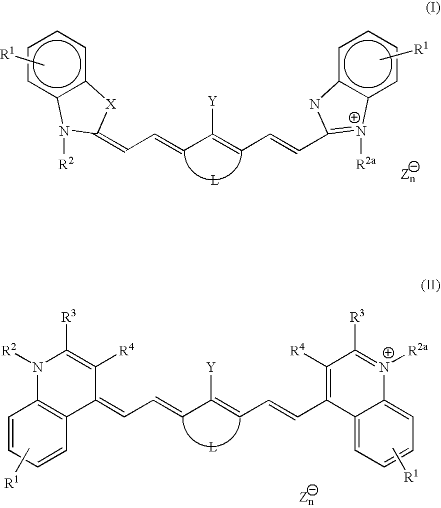
wherein
each R¹ is independently —COOH, —SO₃H, a hydrogen atom, optionally substituted C₁-C₁₂ alkyl, halogen, optionally substituted C₁-C₁₂ alkoxy, —NO₂, —CN, or fused aromatic or heteroaromatic ring systems,
each X is independently —CR³═CR⁴—, —O—, —S—, —NR⁶— or —CR⁵₂—,
each R² is optionally substituted C₁-C₁₂ alkyl, optionally substituted aryl, —(C₁-C₁₂ alkanediyl)-SO₃H or —(C₁-C₁₂ alkanediyl-)COOH,
each R^{2a} is optionally substituted C₁-C₁₂ alkyl, optionally substituted aryl, —(C₁-C₁₂ alkanediyl)-SO₃⁻, —(C₁-C₁₂ alkanediyl)-COO³¹ or —(C₁-C₁₂ alkanediyl)-NR⁶₃⁺,
each R³ and R⁴ are independently —COOH, —SO₃H, —COOR⁶, —CN, —NO₂, —OH, —NR⁶₂, a hydrogen atom, optionally substituted C₁-C₁₂ alkyl, optionally substituted C₁-C₁₂ alkoxy, halogen or aryl,
R⁵ is independently C₁-C₁₂ alkyl,
Z⁻ is Cl⁻, Br⁻, I⁻, SCN⁻, PF₆⁻, SbF₆⁻, AsF₆⁻, aryl-SO₃⁻, alkyl-O—SO₃⁻,
PO₄H₂⁻, CH₃SO₃⁻, CF₃SO₃⁻, (CF₃SO₂)₂N⁻, HSO₄⁻, BF₄⁻ or ClO₄⁻,
n is 0 if R^{2a} is —(C₁-C₁₂ alkanediyl)- SO₃⁻ or —(C₁-C₁ alkanediyl)-COO⁻,
n is 1 if R^{2a} is optionally substituted C₁-C₁₂ alkyl or aryl,
n is 2 if R^{2a} is —(C₁-C₁₂ alkanediyl)-NR⁶₃⁺,
Y is —S—Ar, —Se—Ar—, —O—Ar, —NR⁶—Ar, —SO₂—Ar or —(N-heterocycle),
R⁶ is a hydrogen atom or optionally substituted C₁-C₁₂ alkyl,
Ar is an aromatic group wherein one or more ring carbon atoms are optionally replaced by N, O or S heteroatoms, and the fragment 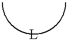
represents C₂-C₃ alkanediyl, optionally substituted with substituents that are the same or different selected from the grouping consisting of one or more C₁-C₁₀ alkyl, C₁-C₁₀ alkoxy, aryl and halogen atoms,
said process comprising the single-step reaction in an inert organic solvent miscible with water of:
(a) a dye of formula (III) 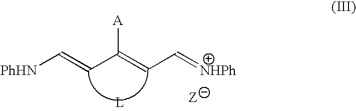
wherein A is Cl or Br and the fragment 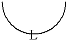
is as defined above for formulas (I) and (II), with
(b) a compound comprising:
(i) a methylene derivative of formulas (IV) or (V) 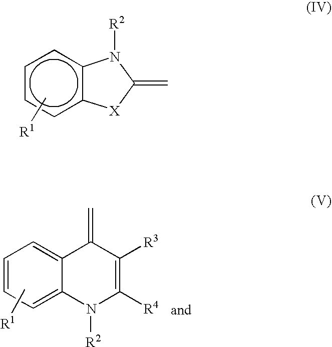
(ii) a quaternary salt of formulas (VI) or (VII), 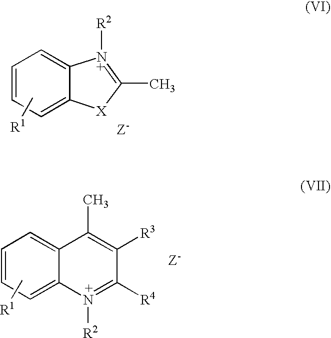
wherein X, R¹, R², R³, R⁴ and Z⁻ are as defined in formulas (I) and (II), and
(c) a compound C comprising:
(i) aromatic and heteroaromatic functionalized compounds Ar—B,
(ii) saturated 5- or 6-membered cyclic amines 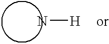
(iii) 5- or 6-membered heteroaromatic compounds 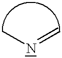
comprising at least one nitrogen atom as heteroatom in the aromatic ring, which nitrogen atom is bonded to the two adjacent ring carbon atoms via a single and a double bond and comprises a free electron pair
wherein
Ar represents 5- or 6-membered aryl, wherein one or more ring carbon atoms are optionally replaced by N, O or S heteroatoms,
B is —NHR⁶, —SH, —OH, —SeH or —SO₂H,
R⁶ is a hydrogen atom or optionally substituted C₁-C₁₂ alkyl, and the saturated cyclic amines optionally comprise an additional N, O or S heteroatom in the ring.

16. The process according to claim 15, wherein the dye (III) is reacted with at least one methylene compound (IV) or at least one quaternary salt (VI) and a compound C, and a cyanine dye of formula (I) is obtained.

17. The process according to claim 15, wherein the dye (III) is reacted with at least one methylene compound (V) or at least one quaternary salt (VII) and a compound C, and a cyanine dye of formula (II) is obtained.

18. The process according to claim 15, wherein the fragment 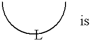
—CH₂-CH₂— or —CH₂-CH₂-CH₂—.

19. The process according to claim 15, wherein Y is —S—Ar.

20. The process according to claim 15, wherein only one methylene derivative or quaternary salt is used and a dye with a symmetrical structure of formulas (I) or (II) is obtained.

21. The process according to claim 15, wherein the compound C and the dye (III) are provided in a reaction vessel and the methylene compound of formulas (IV) or (V) or the quaternary salt of formulas (VI) or (VII) is added in dissolved form.

22. The process according to claim 15, wherein an alkali hydroxide is added to the reaction mixture if B is —SH, —OH, —SeH or —SO₂H.

23. The process according to claim 15, wherein a quaternary salt of formulas (VI) or (VII) is used and an amount of a base equimolar to the amount of quaternary salt is added to the reaction mixture.

24. The process according to claim 15, wherein the cyanine dye of formulas (I) or (II) is precipitated by the addition of a mineral acid.

25. The process according to claim 15, wherein compound C is a aromatic and heteroaromatic functionalized compounds Ar—B.

26. The process according to claim 15, wherein compound C is 5- or 6-membered heteroaromatic compounds 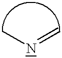
comprising at least one nitrogen atom as heteroatom in the aromatic ring, which nitrogen atom is bonded to the two adjacent ring carbon atoms via a single and a double bond and comprises a free electron pair wherein
Ar represents 5- or 6-membered aryl, wherein one or more ring carbon atoms are optionally replaced by N, O or S heteroatoms,
B is —NHR⁶, —SH, —OH, —SeH or —SO₂H,
R⁶ is a hydrogen atom or optionally substituted C₁-C₁₂ alkyl, and the saturated cyclic amines optionally comprise an additional N, O or S heteroatom in the ring.

27. The process according to claim 15, wherein the cyanine dye of formulas (I) or (II) is subsequently subjected to an extraction.

28. The process according to claim 15, wherein the cyanine dye of formulas (I) or (II) is subjected to an anion exchange.
